(19) United States
(12) Patent Application Publication
(10) Pub. No.: US 2015/0273178 A1
(43) Pub. Date: Oct. 1, 2015

(54) Every day clothing integrated with sensory therapy and on board fidgets

(71) Applicant: Sherryl Lynn Johnson, Colorado Springs, CO (US)

(72) Inventor: Sherryl Lynn Johnson, Colorado Springs, CO (US)

(21) Appl. No.: 14/667,535

(22) Filed: Mar. 24, 2015

Related U.S. Application Data
(60) Provisional application No. 61/995,025, filed on Apr. 1, 2014.

(51) Int. Cl. A61M 21/02

(52) U.S. Cl. CPC A61M 21/02; A61M 2021/0022

(57) ABSTRACT

A clothing system specifically designed with integrated tactile sensory comfort items hereafter referred to as fidgets. Said fidgets are to be integrated with the articles of clothing to be assessed at wearer's convenience. Said system is to be designed discreetly for everyday wear so as to comfort wearers without worry of appearing strange. The clothing may integrate person specific textured patches, hereafter referred to as texture areas, and fidgets for sensory comfort. These may be placed in pockets, as well as inside and outside of shirts, shorts, pants, and sweatshirts and other articles of clothing. Each of the clothing articles may be of different style and size, and may include one or more fidgets. Said clothing articles may also vary in color, shape, and style to allow for a variety of outfits and choices according to wearer appeal and comfort needs.

### CROSS REFERENCE TO RELATED APPLICATIONS

This application claims the benefit of my earlier filed provisional application No. 61/995,025.

### BACKGROUND OF THE INVENTION

Sensory information is received in the brain, but is not always correctly organized into the appropriate responses. The information can become “mixed up” in the brain, which causes inappropriate responses in the situation they often find themselves. Individuals who respond to sensory information differently than most of the general population can find comfort from certain sensory inputs. In addition, almost every individual, under stress, may find comfort in fidgeting.

In one embodiment the present invention may have fidgets integrated directly into the clothing. In an alternative embodiment the clothing may be attached via buttons, hook and loop fastener or other removable means. Studies show that tactile input improves focus, elasticizes the brain, and calms those who seek sensory input.

The present invention integrates these adaptations to help individuals to focus effectively and/or reduce stress. Sensory input devices in the current state of the art are not designed for both comfort and discretion. The fidgets on the clothing articles of the present invention are designed to be discreet yet can also help to calm and comfort the person wearing them.

### CITATIONS

Lavigne J V, Hopkins. “Multi-domain models of risk factors for depression and anxiety symptoms in preschoolers; evidence for common and specific factors.” J Abnorm Child Psychol. (2013) July; 41(5):705-22. Doi; 10.1007/s10802-013-9723-2. PubMed PMID: 23504302. Web. 11 Mar. 2014
Watson, Linda. (Differential Associations between Sensory Response Patterns and Language, Social Communication Measures in Children with Autism or Other Developmental Disabilities.) J Speech Lang Hear Res. Author Manuscript; available in PMC 2012 Dec. 1. Published in final edited form as: J Speech Lang Hear Res. (2011) December; 54(6): 1562-1576. Published online (2011) Aug. 23. Doi: 10.1044/1092-4388(2011-10-0029) PMCID: PMC3325756. Web. 11 Mar. 2014.
Lin Lin, Chein. (Effectiveness of Sensory Processing Strategies on Activity Level in Inclusive Preschool Classrooms.) Neuropsychiatr Dis Treat. 2012; 8: 475-481. Published online (2012) Oct. 23. Doi: 10.2147/NDT.S37146 PMCID: PMC3484897 ArticlPubReaderPDF-173K. Web. 11 Mar. 2014.
Foss-Feig, Jennifer. “TACTILE RESPONSIVENESS PATTERNS AND THEIR ASSOCIATION WITH CORE FEATURES IN AUTISM SPECTRUM DISORDERS.” Res Autism Spectr Disord. Author manuscript; available in PMC 2013 January 1.Published in final edited form as: Res Autism Spectr Disord. (2012) Winter; 6(1): 337-344. Doi: 10.1016/j.rasd.2011.06.007 PMCID: PMC3207504 ArticlePubReaderPDF-301K. Web. 11 Mar. 2014.

### REFERENCED BY

| | Filing | Publica- | | |
|---|---|---|---|---|
| Citing Patent | date | tion date | Applicant | Title |
| US20090260123 | Apr. 17, | Oct. 22, | Eileen Sue | Children's clothing |
| | 2008 | 2009 | Swift | system having a |
| | | | | plurality of clothing |
| | | | | articles and |
| | | | | associated |
| | | | | interchangeable |
| | | | | design elements |
| U.S. Pat. No. | Aug. 5, | Aug. 17, | Mark T. | Weighted exercise |
| 5,937,441 | 1996 | 1999 | Raines | and therapeutic suit |
| U.S. Pat. No. | Oct. 19, | Jan. 18, | Erin M. | A weighted article |
| 7,870,623 | 2007 | 2011 | Judd | for providing deep |
| | | | | pressure therapy |
| | | | | includes a plurality |
| | | | | of patchwork pieces |
| | | | | including a plurality |
| | | | | of layers |
| U.S. Pat. No. | Jun. 10, | Dec. 2, | Sherry J. | Therapy apparel for |
| 6,654,967 | 2002 | 2003 | Haar | children diagnosed |
| | | | | with sensory |
| | | | | integration |
| | | | | dysfunction |

### BRIEF SUMMARY OF THE INVENTION

This invention intends to provide onboard sensory fidgets and texture areas that are designed to provide those individuals who respond to sensory information differently an avenue for relief and comfort. The onboard fidgets can include texture areas, chewing areas, vibrating inserts, weights, olfactory, auditory, and touch stimuli, may be included as per personal comfort appeal and continuous sensory input. These on board fidgets shall attempt to be as discreet as possible so that the individual may enjoy them without feeling awkward or different. These fidgets may be included on a range of clothing items such as: shorts, pants, shirts, sweaters, sweatshirts, shoes, sandals, socks, slippers, sleepwear, leg socks, arm socks, skirts, hats/caps, scarves. Some of the onboard fidgets may be removable and replaceable. The purpose of providing sensory engagement is found in numerous studies to alleviate the symptoms of neurological dysfunction and to reduce stress. (Lavigne J V, Hopkins 705-722) (Watson, 1562-1576) (Lin Lin, 475-481) (Foss-Feig, 337-344)

### BRIEF DESCRIPTION OF THE SEVERAL VIEWS OF THE DRAWINGS

This is a product that is to be used by anyone with sensory needs. This is an article of clothing that will help sooth individuals by giving them immediate tactile stimulation. Texture areas are patches of tactile stimuli such as, but not limited to, burlap, vinyl, carpet, and other such items.

FIG. 1 shows an example of the invention on a hoodie (101) with texture areas on the sleeve (102) and inside of the pockets (103). These texture areas and texture material can be person specific and designed for comfort or stress relief.

FIG. 2 shows an example of an invention on the back of a hoodie with different placements of texture areas on the shoulders (201) and on the sleeves (202) that may each have a different tactile patch according to wearer need. It also depicts fidgets on the lower back of the hoodie (203) and another texture area on the inside of the hoodie (204).

FIG. 3 shows an example of the invention on a pair of pants with a texture area on the inside of the pants (301) and another texture area inside the lower legs of the pants (302). These texture areas are both placed discreetly so they are not visible to an outside observer. An elastic fidget is attached at the belt loop area (303). An example of a detachable fidget is shown in a detached state (304). Also depicted is another knotted fidget attached to the belt loop for convenience (305) and an example of a location where a detachable fidget can be attached (306).

FIG. 4 shows of an example of the invention on the back of a pair of pants with button fidgets on the inside of the back pocket area (401) and another texture area on the inside of the back knee are (402). These are both placed discreetly so they are not obvious comfort items to an outside observer.

FIG. 5 shows an example of the invention on a pair of shorts with a texture area on the inside (501), an elastic fidget placed for convenience near the belt area (502), a knotted fidget (503) and a texture area around the bet loop (504).

FIG. 6 shows an example of the invention on the back of a pair of shorts with a texture area on the inside of the back knee area (601) and button fidgets on the back pocket are (602). These comfort items are both laced discreetly so they are not visible to an outside observer.

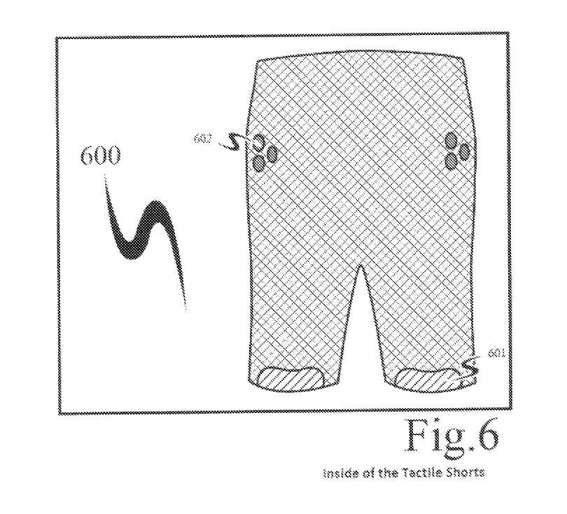

FIG. 7 shows an example of the invention n a t-shirt with texture areas in the inside of the shoulder areas of the shirt (701) and another texture area onside the shirt on the left (702). The figure also shows texture areas on the sleeves (703).

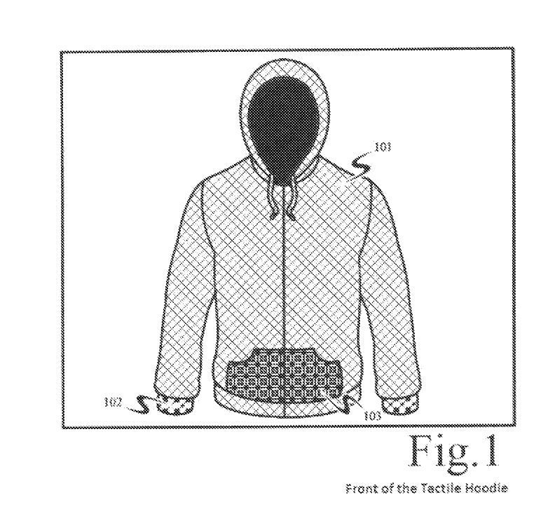

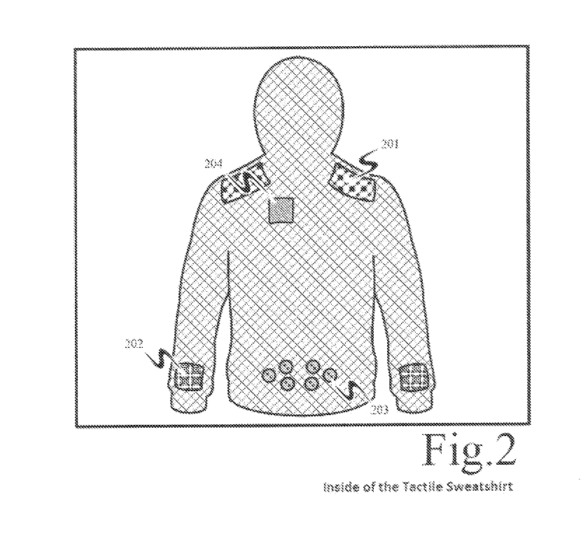

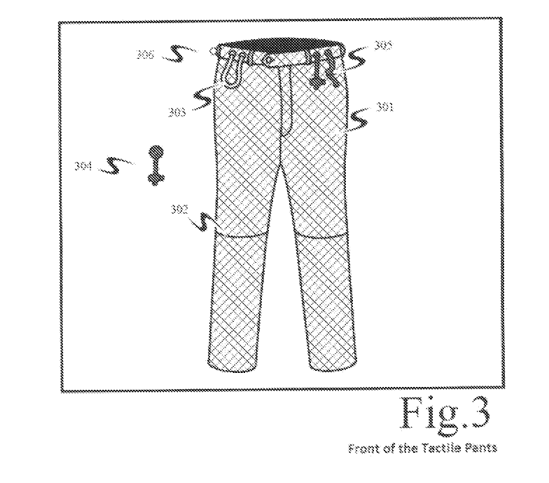

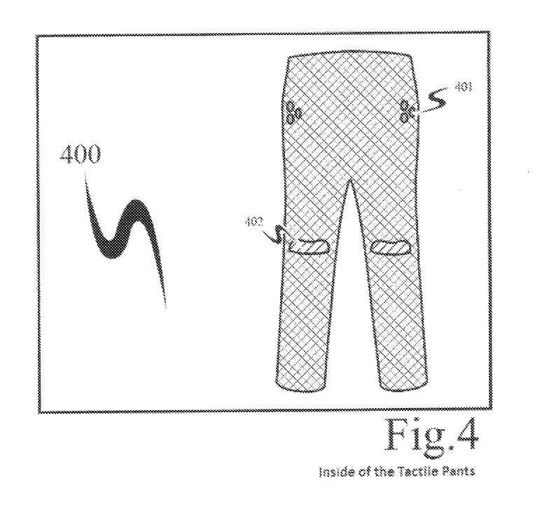

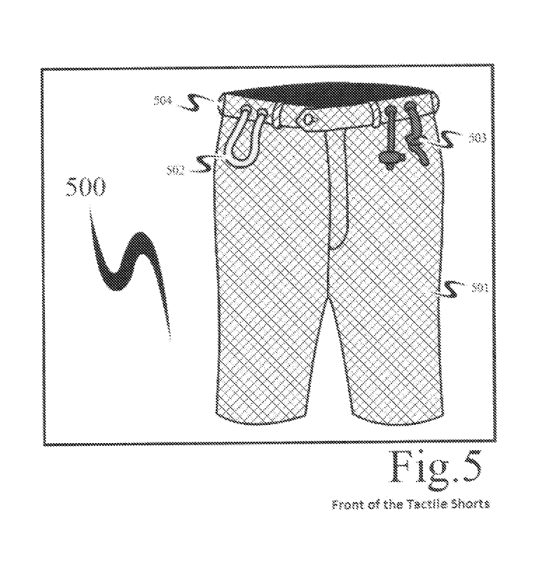

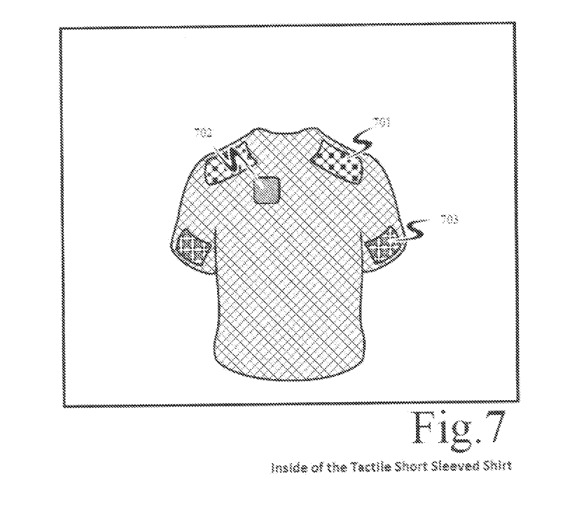

The articles of clothing may vary from anything from a t-shirt (FIG. 7) or hooded sweatshirt (FIG. 1), to jeans, pants (FIG. 3) or shorts (FIG. 5). This provides the capability of integrating texture areas and fidgets into multiple styles of clothing. Fidgets or items such as, but not limited to, sewn on buttons in non-obvious locations (FIG. 2, 202, FIG. 4, 401), knots, and bungee cords at the end of string that can be pulled (FIG. 3, 303, 305), and other such actions to relieve stress and soothe the wearer.

### DETAILED DESCRIPTION OF THE INVENTION

A clothing system having one or more fidgets which we define to be integrated tactile comfort items hereafter referred to as “fidgets”. Fidgets can include, but are not limited to: buttons, coarse fabric patches, zips, pockets for vibrating inserts, pockets for aromatherapy modules. Fidgets are to be integrated with clothing articles worn by individuals having sensory comfort needs. Said fidgets are to be integrated with a plurality of articles of clothing to be accessed by the wearer according to comfort needs during use. The clothing may integrate texture areas which we define to be person specific textured patches, hereafter referred to as “texture areas”. Said articles are to be designed for everyday wear so as to comfort wearers without worry of appearing strange. Each clothing article includes one or more fidgets accessibly located. Clothing articles may vary in size to allow for the wearers' growth and comfort. Said clothing articles may also vary in color, shape and style to allow for a variety of outfits and choices according to wearer appeal. Said clothing articles may also vary as described in paragraph[0015].

It is to be understood that those skilled in the art shall see alternatives to fidgets and texture areas depicted in figures. Fidgets and texture areas in the present invention are integrated discretely with articles of clothing and may take the form of aroma packages, attached vibrating objects, bumpy patches, fuzzy patches, weights, warming areas, zippers, strings, buttons, and other fidgets. Said fidgets and texture areas are designed to provide comfort for individuals with sensory input needs. Said fidgets shall be integrated into clothing discreetly to comfort the wearer in regard to their appearance. Said fidgets and texture areas may vary in size, shape and color to be attached on articles of varying sizes, shapes, styles and colors as discussed below in paragraph[0015].

It is also to be understood that those skilled in the art shall see alternatives to clothing articles depicted in the figures. Clothing articles may take the form of shirts, pants, shorts, socks, hooded sweatshirts, etc. Clothing articles are to be stylized to be worn with other articles of clothing such that an individual may own one article without fidgets or texture areas and may wear it discreetly with an article of the present invention containing fidgets and/or texture areas.

In accordance with the present invention, it is contemplated that those skilled in the art shall see that the clothing articles may be equipped with multiple fidgets to accommodate the range and need of individuals with sensory needs. Those skilled in the art shall also see that fidget locations may also vary. The clothing articles said devices are to be attached to may also vary widely as detailed above in paragraph[0007].

In accordance with the present invention, it is contemplated that those skilled in the art shall see that the clothing articles may be equipped with multiple texture areas to accommodate the range and need of individuals with sensory needs. Those skilled in the art shall also see that texture area location may also vary. Those skilled in the art shall also see that color, size and shape of texture areas may vary. The clothing articles said devices are to be attached to may also vary widely as detailed in paragraph[0015].

In accordance with another aspect of the present invention, it also contemplated that those skilled in the art shall see that clothing articles can be made in any color or size to reasonably accommodate the apparel needs of the wearer. Clothing styles and articles may also vary widely as discussed above in paragraph[0015].

In the ideal form for the present invention clothing articles and fidgets would be machine washable and removable where appropriate, for example to allow the replacement of aromatherapy packets or batteries in a fidget, etc.

Those skilled in the art shall find that the present invention's essence, range and purpose are met in a variety in alterations and modifications. It is to be understood that the present disclosure of the present invention is to cover all such variations within the range and purpose of the present invention and in the following claims and said claims equivalents.

### TECHNICAL FIELD OF THE INVENTION

This application relates to a line of clothing with attached fidgets and tactile sensory areas with the purpose of fulfilling touch stimulation to those who benefit from such activities.

1. A clothing system comprising an article clothing augmented with one or more fidgets designed to give the wearer one or more options for tactile stimulation.

2. The clothing system of claim 1 wherein at least one fidget is directly integrated into said article of clothing.

3. The clothing system of claim 1 wherein at least one fidget is an optional attachment which may be fastened to and removed from said article of clothing.

4. The clothing system of claim 1 wherein the article of clothing and fidget match one another in style.

5. The clothing system of claim 1 wherein at least one fidget is not readily visible to an observer.

6. A tactile sensory stimulus system comprising of one or more tactile sensory augmentation elements integrated with an article of clothing.

7. The tactile sensory stimulus system claim 6 wherein at least one tactile sensory augmentation elements is discreetly integrated into said article of clothing.

8. The tactile sensory stimulus system claim 6 wherein at least one tactile sensory augmentation element is an optional attachment which may be fastened to and removed from said article of clothing.
